(19) United States
(12) Patent Application Publication
(10) Pub. No.: US 2022/0241513 A1
(43) Pub. Date: Aug. 4, 2022

(54) Injection Device with a Filling Level Indicator

(71) Applicant: Sanofi, Paris (FR)

(72) Inventors: Michael Jugl, Frankfurt am Main (DE); Stefan Blancke, Frankfurt am Main (DE)

(21) Appl. No.: 17/620,463

(22) PCT Filed: Jun. 23, 2020
(86) PCT No.: PCT/EP2020/067393

§ 371 Date: Dec. 17, 2021

(30) Foreign Application Priority Data
Jun. 26, 2019 (EP) 19305850.0

(51) Int. Cl. A61M 5/315; A61M 5/24

(52) U.S. Cl. CPC A61M 5/31568; A61M 2005/3126; A61M 5/3155; A61M 5/2422

(57) ABSTRACT

The present disclosure relates to an injection device for setting and injecting of a dose of a medicament. The injection device includes an elongated housing extending along a longitudinal direction and configured to accommodate a cartridge, a first member movably arranged inside the elongated housing for setting of the dose, and a second member, and a piston rod configured to advance in a longitudinal distal direction and operable to exert a distally directed pressure onto a bung of the cartridge for expelling of the dose. Depending on the setting, the first member is rotatable relative to the second member or is rotationally locked to the second member. The injection device includes a filling level indicator threadedly engaged with one of the first member and the second member, the filling level indicator being rotationally locked and longitudinally slidingly engaged with the other one of the first member and the second member.

### CROSS REFERENCE TO RELATED APPLICATIONS

The present application is the national stage entry of International Patent Application No. PCT/EP2020/067393, filed on Jun. 23, 2020, and claims priority to Application No. EP 19305850.0, filed on Jun. 26, 2019, the disclosures of which are incorporated herein by reference.

### TECHNICAL FIELD

The present disclosure relates to the field of injection devices, in particular to hand held injection devices, such as pen-type injectors.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors have to meet a number of user-specific requirements. For instance, with patient's suffering chronic diseases, such like diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure can be configured to be easy to operate, in an unambiguous manner.

Typically, such devices comprise a housing or a particular cartridge holder, adapted to receive a cartridge at least partially filled with the medicament to be dispensed. The device further comprises a drive mechanism, usually having a displaceable piston rod to operably engage with a bung or piston of the cartridge. By means of the drive mechanism and its piston rod, the bung or piston of the cartridge is displaceable in a distal or dispensing direction and may therefore expel a predefined amount of the medicament via a piercing assembly, e.g. in form of an injection needle, which is to be releasably coupled with a distal end section of the housing of the drug delivery device.

The medicament to be dispensed by the drug delivery device may be provided and contained in a multi-dose cartridge. Such cartridges typically comprise a vitreous barrel sealed in distal direction by means of a pierceable seal and being further sealed in proximal direction by the bung. With reusable drug delivery devices an empty cartridge is replaceable by a new one. In contrast to that, drug delivery devices of disposable type are to be entirely discarded when the medicament in the cartridge has been dispensed or used-up.

With some drug delivery devices, such as pen-type injection devices a user has to set a dose of equal or variable size by rotating a dose dial in a clockwise or dose-incrementing direction relative to a body or housing of the injection device. For injecting and expelling of a dose of a liquid medicament the user has to depress a trigger or dose button in a distal direction and hence towards the body or housing of the injection device. Typically, the user uses his thumb for exerting a distally directed pressure onto the dose button, which is located at a proximal end of the dose dial and the dose dial sleeve, while holding the housing of the injection device with the remaining fingers of the same hand.

Injection devices of pen-injector type, such as for instance disclosed in WO 2004/078241 A1 comprise a last dose limiting mechanism. The last dose limiting mechanism may comprise a last dose limiter configured to prevent setting of a dose that would exceed the amount of medicament in the cartridge. For this, the drive sleeve of a drive mechanism could be provided with first and second flanges with an intermediate thread there between. There may be provided a nut disposed between the first and second flanges and keyed to the housing by spline means.

Such a last dose stop limiting mechanism is effective to interrupt a dose setting procedure if a user intends to set a dose that would exceed the residual amount of medicament provided in the cartridge.

Hand held injection devices, such as injection pens typically comprise a transparent cartridge holder configured to receive and/or to accommodate a cartridge filled with the medicament. This allows visual inspection of the medicament located inside the cartridge. Moreover, the transparent cartridge holder also enables visual inspection of the medicament when the cartridge accommodated inside the cartridge holder is also transparent or semi-transparent.

A patient or person intending to set a dose of a predefined size may have difficulties in estimating or precisely determining whether the amount of medicament provided in the cartridge is large enough or sufficient for setting of the required dose. The filling level of the cartridge may be difficult to determine or to inspect visually. Moreover, the cartridge, which may be made of a vitreous material, typically comprises a tubular-shape inherently providing a scattering and/or deflection of light thus making it difficult to precisely determine the amount of medicament inside the cartridge.

### SUMMARY

An injection device can be provided with a filling level indicator operable to visually indicate to a person or patient the residual amount or the remaining filling volume of the cartridge arranged inside the injection device. The injection device can have a precise, robust and reliable filling level indicator visually indicating an actual filling level of the cartridge before a user starts to conduct a dose setting procedure with the injection device. The injection device can have a fluid level indicator that is operable to visually indicate a future fluid level of the cartridge, which the cartridge can have after a dose currently set has been dispensed or expelled from the cartridge. The injection device can designed to enhance patient safety and the overall handling as well as the overall acceptance of handheld injection devices.

In one aspect the disclosure relates to an injection device for setting and injecting of a dose of a medicament. The injection device comprises an elongated housing, e.g., of tubular shape and extending along a longitudinal direction. The elongated housing is configured to accommodate a cartridge. The cartridge comprises a barrel filled with the medicament and is sealed in proximal longitudinal direction by a bung. The bung is displaceable relative to the barrel for expelling of the medicament.

The injection device further comprises a first member movable arranged inside the elongated housing for setting of a dose of the medicament. The injection device further comprises a second member. The second member is a separate member and distinguishes from the first member.

The injection device further comprises a piston rod configured to advance in a longitudinal distal direction. The piston rod is operable to exert a distally directed pressure onto a bung of a cartridge for expelling of the dose. The piston rod is driven by a drive mechanism of the injection device for and during expelling of the dose of the medicament previously set.

During one of setting of the dose and expelling of the dose the first member is rotatable relative to the second member. During the other one of setting of the dose and expelling of the dose the first member is rotationally locked to the second member. The first member and the second member may be engaged in longitudinal direction or in an axial direction. The first member may be immobile and/or positionally locked to the second member with regard to the longitudinal direction or axial direction.

With some examples during and/or for setting of the dose only one of the first member and the second member may be movable while the other one of the first member and the second member is stationary with respect to the housing. During and/or for expelling of the dose the first member and the second member are immobile relative to each other. The first member and the second member may be both immobile relative to the housing, or both, the first member and the second member may be subject to a common or combined movement relative to the housing.

With some other examples, during and/or for setting of the dose the first member and the second member are immobile relative to each other. The first member and the second member may be both immobile relative to the housing, or both, the first member and the second member may be subject to a common or combined movement relative to the housing. At least one of the first member and the second member can be movable relative to the other one of the first member and the second member for and/or during expelling of the dose.

The injection device further comprises a filling level indicator threadedly engaged with one of the first member and the second member. The filling level indicator is rotationally locked and longitudinally slidingly engaged with the other one of the first member and the second member. The filling level indicator may be threadedly engaged with the first member and the filling level indicator may be in a so-called splined engagement with the second member. A rotation of the first member relative to the second member then induces a longitudinal displacement of the filling level indicator relative to the first and the second member.

The filling level indicator is movable in longitudinal direction at least from a start position to an end position relative to the first member and/or relative to the second member. The filling level indicator moves in longitudinal direction towards the end position relative to first member and/or relative to the second member when the first member rotates relative to the second member. The start position and the end position are oppositely located extreme positions of the filling level indicator.

In particular, the filling level indicator moves in longitudinal direction towards the end position relative to first member and/or relative to the second member when the first member rotates relative to the second member along a first direction, e.g. in a dose incrementing direction during dose setting or in a dose decrementing direction during dose expelling.

With some examples the filling level indicator moves in longitudinal direction towards the start position relative to the first member and/or relative to the second member when the first member rotates relative to the second member along a second direction, e.g. in a dose decrementing direction during dose setting, i.e. when a dose already set is too large and wherein a dose previously set is subject to correction prior to dispense or expel the dose.

The filling level indicator is visible from outside the housing when the filling level indicator is in the start position.

Typically, the longitudinal position of the filling level indicator relative to at least one of the first member and the second member directly correlates with a longitudinal position of the piston rod relative to the elongated housing. When the filling level indicator is in the start position the piston rod is in an initial position, typically coinciding with a proximal end position. When the filling level indicator is in the end position the piston rod is in a final or end position typically coinciding with a distal end position. With examples wherein the first member is rotatable relative to the second member only during and/or for setting of the dose, the momentary longitudinal position of the filling level indicator is indicative of a longitudinal position in which the piston rod can be located after dispensing or expelling of the dose actually set. With those examples, wherein the first member is rotatable relative to the second member only during and/or for expelling of the dose the momentary longitudinal position of the filling level indicator relative to at least one of the first member and the second member is directly and instantly indicative of the longitudinal position of the piston rod relative to the housing or cartridge.

In any case the filling level indicator provides a visually perceivable indication being indicative of the momentary or future longitudinal position of the piston rod relative to the housing of the injection device. Since during repeated dose setting and dose expelling procedures the piston rod is and remains in abutment with the bung of the cartridge the longitudinal position of the piston rod is directly indicative of the position of the bung relative to the barrel of the cartridge and hence it is directly indicative of the amount of medicament located in the cartridge.

By making the filling level indicator visible from outside the housing when arranged or located in the start position an indication is provided to a user, that the cartridge has not been used before and that the entirety of the medicament initially provided in the cartridge is still inside the cartridge and is hence still available for dose expelling. Hence, when the filling level indicator is in the start position this is a clear and unequivocal indication to the user or patient of the injection device that the injection device has not been used before for expelling of a dose.

Providing of a filling level indicator in engagement with the first member and the second member and making the filling level indicator visible from outside the housing enables to provide a visual feedback to a user or patient using the injection device that the cartridge is completely filled and that the injection device is in an initial configuration or unused state. Apart from an optional priming shot or priming step when in the start position the filling level indicator is indicative that the initial filling volume of the cartridge is still available.

Generally, the filling level indicator can be provided at any suitable portion of the housing of the injection device. Typically, the filling level indicator could be provided at or near a distal end of a proximal housing component, also denoted as a body of the housing. The body of the housing can be configured to receive or to accommodate the drive mechanism of the injection device.

The housing further comprises a cartridge holder configured to receive or to accommodate the cartridge filled with the medicament. In typical configurations the distal end of the cartridge holder is configured to hold a detachable injection needle. The distal end of the cartridge holder forms or constitutes the distal end of the injection device. The proximal end of the cartridge holder may be interconnected or fastened to a distal end of the body.

At the proximal end of the body there may be provided at least one of a dose dial and an injection button operable to set and/or or to dispense or to expel a dose of e.g. variable size. The filling level indicator may be located at or near a distal end of the body, wherein the body forms or constitutes a proximal portion of the housing of the injection device. The filling level indicator may be located longitudinally offset from a protective cap typically covering the cartridge holder or at least the distal end of the cartridge holder. By providing the filling level indicator on a proximal housing component the momentary filling level of the cartridge that typically corresponds with a momentary longitudinal or axial position of the piston rod relative to the housing can be permanently assessed from outside the housing without the necessity to detach or disassemble the protective cap from the injection device.

Typically and when the filling level indicator is in the start position the piston rod is in a proximal end configuration or proximal end position. In the proximal end configuration or proximal end position the piston rod may be in axial abutment with the bung of the cartridge or the piston rod may be located close to the bung of the cartridge with a well-defined gap there between. In the latter case, the injection device has to undergo a priming procedure before the first injection may take place. During the priming procedure the piston rod is advanced in distal direction until it engages with the bung. This initial but rather small sized distally directed motion of the piston rod may only optionally reflect in a respective modification of the position of the filling level indicator relative to the housing. Also, the filling level indicator may arrive in the start position when the priming procedure has been completed.

As the injection device is repeatedly used for setting and for expelling of a dose of the medicament the piston rod advances successively in longitudinal distal direction. The more the piston rod advances or moves in the longitudinal distal direction the more the filling level indicator advances towards the end position. When the piston rod has reached a distal end position inside the injection device also the filling level indicator has reached the end position relative to the housing.

Typically, the start position of the filling level indicator is a longitudinal start position. The end position of the filling level indicator is a longitudinal end position relative to at least one of the first member and the second member. During setting of the dose or during expelling of the dose the filling level indicator moves towards the end position. With some examples, the filling level indicator may slide relative to the second member along the longitudinal direction. The second member may provide a sliding track for the filling level indicator. During repeated dose setting procedures or during repeated dose expelling procedure the filling level indicator may successively slide along the sliding track as provided by the housing from the start position towards the end position. Typically, the start position is one of a proximal stop position and a distal stop position and the end position is the other one of the proximal stop position and the distal stop position.

According to a further example the filling level indicator is visible from outside the housing when the filling level indicator is in the end position. In this way, the filling level indicator is operable to visually indicate to a user or patient that the cartridge is substantially empty or almost empty and that the residual amount of medicament left in the cartridge is or can be too small or insufficient to set and/or to expel any further dose of the medicament.

In this configuration and when the filling level indicator has reached the end position relative to the housing this is a clear and visual indication to the user or patient, that the present injection device cannot be used any longer for setting and expelling of a dose of the medicament.

According to another example the filling level indicator is visible from outside the housing in any position between the start position and the end position of the filling level indicator. In this way the filling level indicator is operable to visually indicate the amount of the medicament that is provided in the cartridge. Typically, the longitudinal displacement of the filling level indicator during setting of the dose or during expelling of the dose and towards the end position is proportional to a distally directed advancing motion of the piston rod during the dose expelling or dose dispensing procedure. In this way and when the filling level indicator is e.g. located half way between the start position and the end position this is an immediate indication to the user or patient, that half of the initial filling volume of the medicament is still located inside the cartridge.

A proportional mechanical coupling between the filling level indicator, the first member and the second member is of particular benefit to provide a linear visual feedback to the user regarding the momentary filling level of the cartridge located inside the injection device.

Generally and by making the filling level indicator visible from outside the housing in any position between the start position at the end position a rather direct tracking of the accumulated sizes of doses already set and/or dispensed or expelled with the injection device is provided.

According to a further example the first member and the second member comprise a first stop and a second stop. Here, one of the first member and the second member may comprise both, the first stop and the second stop. Alternatively, one of the first member and the second member comprises the first stop and the other one of the first member and the second member comprises the second stop.

The first stop and the second stop are separated from each other along the longitudinal direction. Typically, the first stop defines the start position of the filling level indicator. The second stop typically defines the end position of the filling level indicator. When in the start position the filling level indicator may be engaged with the first stop. In the start position the filling level indicator may be in abutment with the first stop. The filling level indicator may be in at least one of an axial or tangential abutment with the first stop when in the start position.

In a further example the filling level indicator engages the second stop when arriving or when in the end position. Here, the filling level indicator is operable to prevent a further movement of the first member relative to the second member at least with regard to one direction, e.g. the first direction of movement when the filling level indicator is engaged or is abutment with the second stop. Here, the second stop forms or provides a last dose stop effective to prevent setting of a dose that would exceed the residual amount of medicament provided in the cartridge.

Accordingly, the second stop of the first member or the second member defines the end position of the filling level indicator relative to the housing. The filling level indicator can be in at least one of an axial or tangential engagement with the second stop when in the end position.

In this way and as the first member is subject to a rotational movement relative to the second member the filling level indicator starts to move according to a helical path relative to that one of the first and the second member to which the filling level indicator is threadedly engaged. At the same time the filling level indicator starts to slide along a sliding track provided by the other one of the first member and the second member. With reaching or when in one of the start position and the end position the filling level indicator engages with a respective stop. When reaching the start position the filling level indicator engages, e.g. abuts with the first stop. When reaching the end position the filling level indicator can engage, e.g. abut with the second stop.

At least one of the first stop and the second stop can be operable to a further displacement of the filling level indicator beyond the first stop or second stop, respectively. Since the filling level indicator is rotationally locked to one of the first and the second members a circumferential, tangential or longitudinal abutment between the filling level indicator with one of the first stop and the second stop of one of the first member and the second member immediately blocks any further rotation of the first member relative to the second member, e.g. along the first direction, which might a dose incrementing direction when the first member and the second member are subject to a relative movement only during setting of the dose.

In this way, the filling level indicator is not only operable to visualize a momentary position of the piston rod but can be also operable to prevent setting of a dose that would exceed the amount of medicament provided in the cartridge.

With some examples, the second member is a housing component, e.g. a body of the elongated housing and first member is one of a dose setting member and a dose expelling member, wherein the dose setting member is subject to a rotational movement relative to the housing component or body only during dose setting and wherein the dose expelling member is subject to a rotational movement relative to the housing component or body only during dose expelling.

With some examples the first member comprises a sleeve-like or tubular shape. Typically, and when the first member is implemented as a dose setting member or as a dose expelling member it is arranged inside the second member, which may comprise a tubular shaped hollow body. The first member and the second member may be arranged coaxially in a nested or convoluted way. An outside surface of the first member may be in engagement with an inside portion of the filling level indicator. An outside facing portion of the filling level indicator is then typically engaged with an inside facing portion of the second member, hence with the body.

When the first member is threadedly engaged with the filling level indicator the second member is splined with the filling level indicator. During setting of the dose the first member may rotate relative to the second member thus leading to a longitudinal motion of the filling level indicator towards the end position when the first member is rotated relative to the second member along the first direction. During dose expelling the first member is non-rotationally locked to the second member, e.g. by way of a clutch. Then and during dose expelling the first member may be either stationary with regard to the housing or body or it may be subject to a non-rotational purely longitudinal sliding motion. In the latter case, both the filling level indicator and the first member may be subject to a common and/or combined sliding motion relative to the housing.

According to another example the housing of the injection device further comprises an elongated aperture extending along the longitudinal direction. Moreover, the filling level indicator comprises an indicating portion. The indicating portion is visible through the elongated aperture of the housing. The scale typically extends along a sliding track or displacement path of the filling level indicator starting from the start position and ending at the end position. With some examples the elongated aperture is directly provided in the first member.

With typical examples, the elongation of the aperture is as long as the distance from the start position to the end position of the filling level indicator. The total longitudinal elongation of the aperture may be indicative of and may directly correspond to the maximum or initial filling volume of the cartridge of the injection device. The longitudinal position of the filling level indicator and/or the longitudinal position of the indicating portion of the filling level indicator relative to the oppositely located longitudinal end of the elongated aperture is directly indicative of the amount of the medicament provided in the cartridge in relation to the initial or total filling volume of the cartridge.

With a further example the elongated aperture of the housing comprises a first longitudinal end further comprises an oppositely located second longitudinal end. When the filling level indicator is in the start position the indicating portion of the filling level indicator may be located close to or may be in abutment, e.g. in a longitudinal and hence axial abutment with the first longitudinal end of the elongated aperture. When the filling level indicator is in the end position the respective indicating portion of the filling level indicator may be located close to the second longitudinal end of the elongated aperture. It may overlap with the second longitudinal end and/or it may abut, e.g. axially abut with the second longitudinal end of the aperture.

In this way, at least the second longitudinal end of the aperture may provide a stop to engage with the longitudinally displaceable filling level indicator. When the filling level indicator reaches the second end position it may axially engage, i.e. it may axially abut with the second longitudinal end of the elongated aperture. In this way, the second longitudinal end of the elongated aperture may serve as the second stop as describes above. It may provide a last dose stop configured to prevent any further longitudinal motion of the filling level indicator beyond the end position relative to the housing.

According to a further example the elongated aperture is covered or closed by a transparent window. The elongated aperture may be entirely closed or filled with the transparent window. In this way, the interior of the housing of the injection device can be effectively protected against ingress of humidity and/or particles. Moreover, a manual manipulation of the filling level indicator can be effectively prevented by the transparent window. The transparent window covering the elongated aperture may entirely cover and/or enclose the filling level indicator located underneath.

According to a further example the housing comprises an elongated aperture extending along the longitudinal direction of the elongated housing. At least an indicating portion of the filling level indicator is located outside the housing whereas a residual or base portion of the filling level indicator is located inside the housing, in particular in a region between the interior of the elongated housing and an exterior of one of the first member or second member.

By providing at least a dedicated portion of the filling level indicator outside the housing the legibility and size of the filling level indicator, in particular the legibility and the size of the indicating portion of the filling level indicator can be increased compared to an example, wherein the filling level indicator is entirely located inside the housing. Moreover, arranging of at least a portion of the filling level indicator on or at an outside the housing may even provide a haptic feedback to a user regarding the momentary filling level of the cartridge. The position of the indicating portion of filling level indicator could be haptically sensed, it may become palpable. In addition, by providing at least the indicating portion of the filling level indicator on or at the outside of the housing the indicating portion operable to indicate a momentary longitudinal position of the piston rod relative to the housing can be spatially separated from a base portion of the filling level indicator at least with regard to one of the longitudinal or circumferential direction. The base portion mechanically engages both, the first member and e.g. an inside sidewall portion of the second member, e.g. of the elongated housing.

According to a further example the filling level indicator comprises a L-shaped rod comprising the indicating portion and further comprising a bridging portion. Here, the bridging portion extends through the aperture and is connected or connectable to a base portion of the filling level indicator. The base portion of the filling level indicator is mechanically engaged with both, the first member and the second member. The bridging portion extending through the aperture comprises a radial extension that is larger than the thickness of the sidewall of the elongated housing. In this way, the L-shaped rod of the filling level indicator and in particular the indicating portion may comprise one of an annular circumferentially extending structure and a longitudinally extending rod-shaped structure provided on the outside of the housing.

With an elongated or longitudinally extending indicating portion the L-shaped rod enables an arrangement of the filling level indicator, at least of an indicating portion of the filling level indicator axially or longitudinally offset from the base portion of the filling level indicator. In this way, that portion of the filling level indicator being visually perceivable from outside the housing can be located at an axial and/or or circumferential offset from the first member and/or from the aperture in the housing.

In this way, the filling level can be indicated at almost any arbitrary or desirable position on the outside surface of the housing of the injection device. Accordingly, the overall design of the injection device can be adapted and designed to conform given user requirements.

According to a further example the injection device comprises an outer housing covering or enclosing at least a portion of the housing in the region of the filling level indicator. The outer housing comprises a transparent window overlapping at least with the indicating portion of the filling level indicator. The outer housing is particularly intended for such examples, wherein the aperture in the housing or inner housing of the injection device is void of a transparent window and wherein the bridging portion of the filling level indicator extends through the aperture of the housing or inner housing. By way of the outer housing the aperture of the inner housing through which the filling level indicator extends can be effectively closed or encapsulated and hence protected against ingress of particles or humidity.

According to another example the housing of the injection device comprises a cartridge holder that is configured to accommodate the cartridge. In this particular example the cartridge holder is opaque. It is opaque at least in view of the visible spectrum of electromagnetic radiation, e.g. visible light. With other examples the cartridge holder is opaque at least for a dedicated spectrum of electromagnetic radiation. The cartridge holder may be opaque with regards to ultraviolet radiation and/or with regards to infrared radiation.

Apart from a through opening in a distal face for receiving a proximal tip of an injection needle the cartridge holder is void of any apertures or through openings in a sidewall. In this way, the cartridge holder is configured to completely enclose the cartridge and to protect the cartridge against electromagnetic radiation from outside.

Providing of an opaque cartridge holder is beneficial to protect the medicament located therein against respective electromagnetic radiation. In this way, the cartridge holder serves to protect the cartridge and the medicament located therein against detrimental effects arising from illumination with one of electromagnetic radiation in the visible spectral range, in the ultra violet spectral range and/or in the infrared spectral range.

Some medicaments are rather sensitive to electromagnetic radiation. Here, the opaque and/or non-transparent cartridge holder is beneficial to protect the medicament against such detrimental environmental influences.

With the filling level indicator a visual inspection of the cartridge and/or of the medicament located thereon may become superfluous. Implementation of the filling level indicator thus enables to make use of a cartridge holder being substantially opaque at least with regards to one of infrared radiation, light in the visible spectral range and UV radiation.

According to another example the housing comprises a scale extending along the elongated aperture of the housing. The indicating portion of the filling level indicator comprises a pointer that points towards the scale. The scale typically comprises numerous marks or symbols indicating the longitudinal position of the piston rod relative to the housing and thus indicating the residual amount of medicament left in the cartridge. The scale may comprise numerous ticks or marks. Such ticks or marks may be arranged equidistantly in longitudinal direction along the longitudinal extent of the scale.

The scale may be provided with symbols or numbers thus indicating the amount of the medicament that is still available in the cartridge.

Typically, the pointer comprises a pointed tip pointing towards the scale. The pointer may be provided as a further mark on an outside surface of the indicating portion of the filling level indicator. The scale may be provided on the outside of the housing. It may be divided into numerous sections that are located longitudinally adjacent. Each one of the sections may be characteristic for a particular configuration of the injection device. A section adjacent to the first longitudinal end of the aperture may be indicative that the cartridge is almost or substantially completely filled with the medicament. A further section located at the second longitudinal end of the aperture may be indicative that less than a predefined amount of medicament is located in the cartridge when the filling level indicator overlaps with this particular section of the scale.

Numerous and/or adjacently located sections of the scale may be provided with different colors, thus enabling a rather intuitive and comprehensive understanding of a momentary configuration of the injection device.

According to another example the filling level indicator comprises a threaded section and at least one of a radial protrusion or radial recess. The threaded section is an outer threaded section or an inner threaded section. The threaded section is threadedly engaged with a correspondingly shaped counter threaded section of one of the first member and the second member. The at least one of the radial protrusion or radial recess of the filling level indicator is in a longitudinal sliding engagement with a correspondingly shaped radial recess or radial protrusion of the other one of the first member and the second member. The mutually corresponding recess(es) and protrusion(s) of one of the first or second member and the filling level indicator enable(s) a longitudinally directed sliding motion of the filling level indicator relative to the respective first or second member provided with the correspondingly shaped radial recess or radial protrusion.

There may be provided at least one radial recess or radial protrusion on the filling level indicator engaged and/or mating with the correspondingly shaped radial protrusion or recess of one of the first member and the second member. There may be provided numerous radial recesses and/or protrusions on the filling level indicator engaged and/or mating with a corresponding number of correspondingly shaped radial protrusions and/or recesses of one of the first member and the second member. In this way a twofold, threefold or even fourfold sliding and rotationally locked engagement between the filling level indicator and one of the first and second members can be provided, thus leading to a rather robust and reliable implementation of the filling level indicator.

With one example the filling level indicator comprises an inner thread engaged with an outer thread of the second member. The filling level indicator further comprises a radially outwardly extending protrusion engaged with a correspondingly shaped radial and longitudinally extending recess or groove provided on an inside surface of the first member. In this way and as the second member, e.g. implemented as a dose setting sleeve or drive sleeve of a drive mechanism, is rotated relative to the housing the filling level indicator slides along the longitudinal recess(es) or groove(s) of the first member. Here, the first member may be implemented as a housing or body of e.g. tubular shape. The first member may be also implemented as an insert rigidly attached and/or fixed to or inside the body or housing. Here, the longitudinally extending recess or a groove of the first member provides a sliding track extending from the start position of the filling level indicator towards and/or up to the end position of the filling level indicator relative to the first member, e.g. implemented as a body or a housing.

With other examples the filling level indicator comprises an outer thread engaged with a correspondingly shaped inner thread of the first member. Here, the filling level indicator comprises one of a radially inwardly extending protrusion or a respective recess to engage with a correspondingly shaped recess or radially extending protrusion of the second member. The second member, e.g. implemented as a dose setting sleeve or drive sleeve, may comprise one of an elongated and longitudinally extending groove in sliding engagement with a radially extending protrusion of the filling level indicator. Alternatively, it is the filling level indicator that comprises an elongated groove or elongated radial recess engaged with a correspondingly shaped radial protrusion of the second member.

The radial protrusion is one of a radially outwardly and a radially inwardly extending protrusion. The longitudinal recess is complementary shaped to the radial protrusion. When the radial protrusion extends radially outwardly on the filling level indicator, the elongated recess can be provided on an inside sidewall portion of the first member or the second member. When the radial protrusion extends radially inwardly on the filling level indicator, the elongated recess can be provided on an outside sidewall portion of the first member or the second member.

With either case and when the second member is subject to a rotational movement relative to the first member the filling level indicator is subject to a longitudinal displacement relative to the first member in the direction towards the end position.

When in a splined and hence longitudinally sliding engagement with the second member the filling level indicator may comprise an arc-shaped or semicircular structure, thus representing a half nut. When the filling level indicator is in a longitudinal and/or splined sliding engagement with the first member and when the filling level indicator is threadedly engaged with the second member the filling level indicator can be of annular shape. Here, the second member, e.g. implemented as a housing, body or housing component, and comprising the longitudinally extending elongated aperture provides unaltered visual inspection of the longitudinal and/or axial position of the filling level indicator in the aperture of the second member, and hence from outside the housing.

According to a further example the first member is rotationally locked to the housing during dispensing of a dose of the medicament while the piston rod advances in the longitudinal distal direction relative to the housing. During dose dispensing or dose expelling the first member may be non-movable relative to the second member. The first member may be locked to the second at least with regard to a rotational movement. The first member may be also locked to the second member with regards to an axial or longitudinal movement. The first member may be permanently locked in axial direction relative to the second member, i.e. during both, dose setting and dose dispensing or dose expelling.

In another example the filling level indicator is only and exclusively displaceable relative to at least one of the first member and the second member during setting of the dose. During and/or for dose setting, the filling level indicator may be displaceable relative to both, the first member and the second member. The filling level indicator may be subject to a sliding motion relative to the second member during setting of the dose. The filling level indicator may be subject to a rotational or helical motion relative to the first member during setting of the dose. As a dose is set the filling level indicator and the first member are rotatable relative to each other. They may be kept non-rotatable relative to each or may be rotationally locked to each other when the dose is expelled. In this way and when the filling level indicator is moved along the sliding track as defined by the first member during dose setting the momentary position of the filling level indicator relative to the first member and/or relative to the elongated aperture thereof is indicative and/or represents the summarized doses previously set by the injection device.

When the filling level indicator is in the end position the summarized set doses that have been previously set by the injection device typically equals the initial amount of medicament inside the cartridge.

The injection device typically comprises a switchable clutch selectively engageable with the first member and the second member. Typically, and when the switchable clutch is in one of a dose setting state and a dose expelling state the first member is rotatable relative the second member. When the clutch is switched into the other one of the dose setting state and the dose expelling state the first member is and/or becomes rotationally locked to the second member. With some examples, the first member is rotatable relative to the second member when the clutch is in the dose setting state. The first member is rotationally locked to the second member when the clutch is in the dose expelling state.

With some other examples, the first member is rotatable relative to the second member when the clutch is in the dose expelling state. The first member is rotationally locked to the second member when the clutch is in the dose setting state.

One implementation of the present disclosure is defined by the content of the claims. The injection device is not limited to specific embodiments or examples but comprises any combination of elements of different embodiments or examples. The present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The term “drug” or “medicament”, as used herein, means a pharmaceutical formulation containing at least one pharmaceutically active compound, wherein in one embodiment the pharmaceutically active compound has a molecular weight up to 1500 Da and/or is a peptide, a protein, a polysaccharide, a vaccine, a DNA, a RNA, an enzyme, an antibody or a fragment thereof, a hormone or an oligonucleotide, or a mixture of the above-mentioned pharmaceutically active compound, wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis, wherein in a further embodiment the pharmaceutically active compound comprises at least one peptide for the treatment and/or prophylaxis of diabetes mellitus or complications associated with diabetes mellitus such as diabetic retinopathy, wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g., a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by A and K. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H-H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE FIGURES

In the following, numerous examples of the injection device comprising a filling level indicator will be described in greater detail by making reference to the drawings, in which:

FIG. 1 shows an example of an injection device,

FIG. 2 shows the injection device of FIG. 1 in an exploded perspective view,

FIG. 3 shows a cross-section through the injection device,

FIG. 4 is an enlarged view of a portion of FIG. 3 with the filling level indicator in an initial configuration,

FIG. 5 is an illustration corresponding to FIG. 4 with the filling level indicator in an intermediate configuration,

FIG. 6 is an enlarged view of a portion of the housing of the injection device according to FIG. 4 as seen from outside

FIG. 7 is an enlarged view of a portion of the housing of the injection device according to FIG. 5 as seen from outside

FIG. 8 is a transverse cross-section through the filling level indicator engaged with the housing,

FIG. 9 is a longitudinal cross-section through a further example of the filling level indicator and

FIG. 10 is a transverse cross-section through the filling level indicator according of FIG. 9.

### DETAILED DESCRIPTION

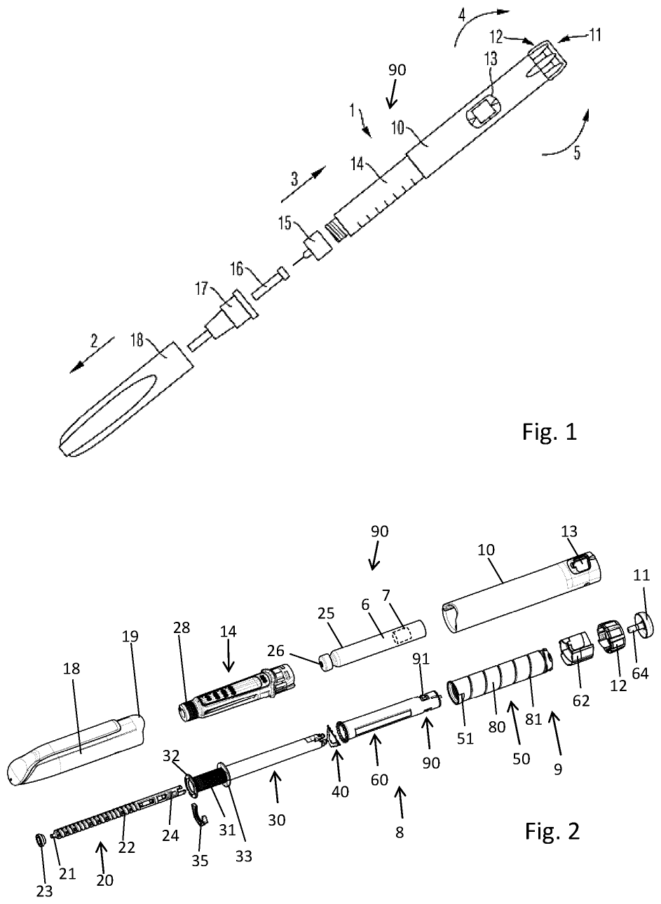

One of a plurality of examples of an injection device 1 suitable for implementation of a filling level indication of a cartridge is shown in FIGS. 1 and 2. The injection device 1 is a pre-filled disposable injection device that comprises a housing 90 to which an injection needle 15 can be affixed. The housing 90 comprises a body 10 forming a proximal end of the housing and a cartridge holder 14 forming a distal end of the injection device 1. The injection needle 15 is protected by an inner needle cap 16 and either an outer needle cap 17 and/or a protective cap 18 that is configured to enclose and to protect a distal section of the housing 90 of the injection device 1. The housing 90 comprises the body 10 configured to accommodate a drive mechanism 8 as shown in FIG. 2. The distal housing component is commonly denoted as a cartridge holder 14. The cartridge holder 14 may be permanently or releasably connected to the body 10.

The body 10 may form or constitute a main body or main housing of the injection device 10. Furthermore, the body 10 may form or constitute a second member according to the terminology of the present disclosure. The cartridge holder 14 can be configured to accommodate a cartridge 6 that is filled with a liquid medicament. The cartridge 6 comprises a cylindrically shaped or tubular-shaped barrel 25 sealed in proximal direction 3 by a bung 7 located inside the barrel 25. The bung 7 is displaceable relative to the barrel 25 of the cartridge 6 in a distal direction 2 by a piston rod 20. A distal end of the cartridge 6 is sealed by a pierceable seal 26 configured as a septum and being pierceable by a proximally directed tipped end of the injection needle 15. The cartridge holder 14 comprises a threaded socket 28 at its distal end to threadedly engage with a correspondingly threaded portion of the injection needle 15. By attaching the injection needle 15 to the distal end of the cartridge holder 14 the seal 26 of the cartridge 6 is penetrated thereby establishing a fluid transferring access to the interior of the cartridge 6.

When the injection device 1 is configured to administer e.g. human insulin, the dosage set by a dose dial 12 at a proximal end of the injection device 1 may be displayed in so-called international units (IU), wherein 1 IU is the biological equivalent of about 45.5 μg of pure crystalline insulin (1/22 mg). The dose dial 12 may be rotatable relative to the housing 90 or body 10 with regard to a longitudinal axis.

As shown further in FIGS. 1 and 2, the body 10 comprises a dosage window 13 that may be in the form of an aperture in the body 10. The dosage window 13 permits a user to view a limited portion of a number sleeve 80 that is configured to move when the dose dial 12 is turned, to provide a visual indication of a currently set dose. The dose dial 12 is rotated on a helical path with respect to the body 10 when turned during setting and/or dispensing or expelling of a dose.

The injection device 1 may be configured so that turning the dosage knob or dose dial 12 causes a mechanical click sound to provide acoustical feedback to a user. The number sleeve 80 mechanically interacts with a piston in the insulin cartridge 6. When the needle 15 is stuck into a skin portion of a patient, and when the trigger 11 or injection button is pushed, the insulin dose displayed in display window 13 can be ejected from injection device 1. When the needle 15 of the injection device 1 remains for a certain time in the skin portion after the trigger 11 is pushed, a high percentage of the dose is actually injected into the patient's body. Ejection of an insulin dose may also cause a mechanical click sound, which is however different from the sounds produced when using the dose dial 12.

In this embodiment, during delivery of the insulin dose, the dose dial 12 is turned to its initial position in an axial movement, that is to say without rotation, while the number sleeve 80 is rotated to return to its initial position, e.g. to display a dose of zero units.

The injection device 1 may be used for several injection processes until either the cartridge 6 is empty or the expiration date of the medicament in the injection device 1 (e.g. 28 days after the first use) is reached.

Furthermore, before using injection device 1 for the first time, it may be necessary to perform a so-called “prime shot” to remove air from the cartridge 6 and the needle 15, for instance by selecting two units of the medicament and pressing trigger 11 while holding the injection device 1 with the needle 15 upwards. For simplicity of presentation, in the following, it will be assumed that the ejected amounts substantially correspond to the injected doses, so that, for instance the amount of medicament ejected from the injection device 1 is equal to the dose received by the user.

An example of the drive mechanism 8 is illustrated in more detail in FIG. 2. It comprises numerous mechanically interacting components. A flange like support of the body 10 comprises a threaded axial through opening threadedly engaged with a first thread or distal thread 22 of the piston rod 20. The distal end of the piston rod 20 comprises a bearing 21 on which a pressure foot 23 is free to rotate with the longitudinal axis of the piston rod 20 as an axis of rotation. The pressure foot 23 is configured to axially abut against a proximally facing thrust receiving face of the bung 7 of the cartridge 6. During a dispensing action the piston rod 20 rotates relative to the body 10 thereby experiencing a distally directed advancing motion relative to the body 10 and hence relative to the barrel 25 of the cartridge 6. As a consequence, the bung 7 of the cartridge 6 is displaced in distal direction 2 by a well-defined distance due to the threaded engagement of the piston rod 20 with the body 10.

The piston rod 20 is further provided with a second thread 24 at its proximal end. The distal thread 22 and the proximal thread 24 are oppositely handed.

The injection device 1 includes a dose setting member 30, e.g. forming or constituting a drive sleeve. The dose setting member 30 or drive sleeve may form or constitute a first member according to the terminology of the present disclosure. Here, the first member 30, e.g. in form of the dose setting member 30 or drive sleeve, has a hollow interior to receive the piston rod 20.

The dose setting member 30 comprises an inner thread which is threadedly engaged with the proximal thread 24 of the piston rod 20. Moreover, the dose setting member 30 comprises an outer threaded section 31 at its distal end. The threaded section 31 is axially confined between a first stop 32 and a second stop 33. The first stop 32 comprises distal flange portion. The second stop 33 comprises a proximal flange portion located at a predefined axial distance from the distal flange portion 32. Between the two flange portions 32, 33 there is provided a filling level indicator 35. In the present example the filling level indicator 35 comprises a semi-circular nut having an internal thread 38 mating the threaded section 31 of the dose setting member 30. The filling level indicator 35 may also form or constitute a last dose limiter e.g. in form of a last dose nut.

The filling level indicator 35 further comprises a radial recess or protrusion 39 at its outer circumference to engage with a complementary-shaped recess 99 or protrusion at an inside of the sidewall of the body 10. In this way the filling level indicator 35 is splined to the body 10. A rotation of the dose setting member 30 in a dose incrementing direction 4 or clockwise direction during consecutive dose setting procedures leads to an accumulative axial displacement of the filling level indicator 35 relative to the dose setting member 30. There is further provided an annular spring 40 that is in axial abutment with a proximally facing surface of the flange portion 33. Moreover, there is provided a tubular-shaped clutch 60. At a first end the clutch 60 is provided with a series of circumferentially directed saw teeth. Towards a second opposite end of the clutch 60 there is located a radially inwardly directed flange.

Furthermore, there is provided a dose dial sleeve also denoted as number sleeve 80. The number sleeve 80 is provided outside of the spring 40 and the clutch 60 and is located radially inward of the body 10. A helical groove 81 is provided about an outer surface of the number sleeve 80. The body 10 is provided with the dosage window 13 through which a part of the outer surface of the number 80 can be seen. The body 10 is further provided with a helical rib at an inside sidewall portion of an insert piece 62, which helical rib is to be seated in the helical groove 81 of the number sleeve 80. The tubular shaped insert piece 62 is inserted into the proximal end of the body 10. It is rotationally and axially fixed to the body 10. There are provided first and second stops on the body 10 to limit a dose setting procedure during which the number sleeve 80 is rotated in a helical motion relative to the body 10.

The dose dial 12 in form of a dose dial grip is disposed about an outer surface of the proximal end of the number sleeve 80. An outer diameter of the dose dial 12 typically corresponds to and matches with the outer diameter of the body 10. The dose dial 12 is secured to the number 80 to prevent relative movement there between. The dose dial 12 is provided with a central opening.

The trigger 11, also denoted as dose button is substantially T-shaped. It is provided at a proximal end of the injection device 10. A stem 64 of the trigger 11 extends through the opening in the dose dial 12, through an inner diameter of extensions of the dose setting member 30 and into a receiving recess at the proximal end of the piston rod 20. The stem 64 is retained for limited axial movement in the dose setting member 30 and against rotation with respect thereto. A head of the trigger 11 is generally circular. The trigger side wall or skirt extends from a periphery of the head and is further adapted to be seated in a proximally accessible annular recess of the dose dial 12.

To dial a dose a user rotates the dose dial 12. With the spring 40 also acting as a clicker and the clutch 60 engaged, the dose setting member 30, the spring or clicker 40, the clutch 60 and the number sleeve 80 rotate with the dose dial 12. Audible and tactile feedback of the dose being dialed is provided by the spring 40 and by the clutch 60. Torque is transmitted through saw teeth between the spring 40 and the clutch 60. The helical groove 81 on the number sleeve 80 and a helical groove in the dose setting member 30 have the same lead. This allows the number sleeve 80 to extend from the body 10 and the dose setting member 30 to climb the piston rod 20 at the same rate. At a limit of travel a radial stop on the number sleeve 80 engages either with a first stop or a second stop provided on the body 10 to prevent further movement in a first sense of rotation, e.g. in a dose incrementing direction 4. Rotation of the piston rod 20 is prevented due to the opposing directions of the overall and driven threads on the piston rod 20.

The filling level indicator 35 keyed to the body 10 is advanced along the threaded section 31 by the rotation of the dose setting member 30. When a final dose dispensing position is reached, a radial stop formed on a surface of the filling level indicator 35 may abut a radial stop on the second stop 33 or proximal flange portion of the dose setting member 30, preventing both, the filling level indicator 35 and the dose setting member 30 from rotating further.

Should a user inadvertently dial beyond the desired dosage, the injection device 1, configured as a pen-injector allows the dosage to be dialed down without dispense of the medicament from the cartridge 6. For this the dose dial 12 is simply counter-rotated. This causes the system to act in reverse. A flexible arm of the spring or clicker 40 then acts as a ratchet preventing the spring 40 from rotating. The torque transmitted through the clutch 60 causes the saw teeth to ride over one another to create the clicks corresponding to dialed dose reduction. Typically, the saw teeth are so disposed that a circumferential extent of each saw tooth corresponds to a unit dose. Here, the clutch may serve as a ratchet mechanism.

As an alternative or in addition the ratchet mechanism may comprise at least one ratchet feature, such as a flexible arm on the sidewall of the tubular-shaped clutch 60. The at least one ratchet feature may comprise a radially outwardly extending protrusion e.g. on a free end of the flexible arm. The protrusion is configured to engage with a correspondingly shaped counter ratchet structure on an inside of the number sleeve 80. The inside of the number sleeve 80 may comprise longitudinally shaped grooves or protrusions featuring a saw-tooth profile. During dialing or setting of a dose the ratchet mechanism allows and supports a rotation of the number sleeve 80 relative to the clutch 60 along a second sense of rotation 5, which rotation is accompanied by a regular clicking of the flexible arm of the clutch 60. An angular momentum applied to the number sleeve 80 along the first sense of rotation for is unalterably transferred to the clutch 60. Here, the mutually corresponding ratchet features of the ratchet mechanism provide a torque transmission from the number sleeve 80 to the clutch 60.

When the desired dose has been dialed the user may simply dispense the set dose by depressing the trigger 11. This displaces the clutch 60 axially with respect to the number sleeve 80 causing dog teeth thereof to disengage. However, the clutch 60 remains keyed in rotation to the dose setting member 30. The number sleeve 80 and the dose dial 12 are now free to rotate in accordance with the helical groove 81.

The axial movement deforms the flexible arm of the spring 40 to ensure the saw teeth cannot be overhauled during dispense. This prevents the dose setting member 30 from rotating with respect to the body 10 though it is still free to move axially with respect thereto. The deformation is subsequently used to urge the spring 40 and the clutch 60 back along the dose setting member 30 to restore the connection between the clutch 60 and the number sleeve 80 when the distally directed dispensing pressure is removed from the trigger 11.

The longitudinal axial movement of the dose setting member 30 causes the piston rod 20 to rotate through the through opening of the support of the body 10, thereby to advance the bung 7 in the cartridge 6. Once the dialed dose has been dispensed, the number sleeve 80 is prevented from further rotation by contact of at least one stop extending from the dose dial 12 with at least one corresponding stop of the body 10. A zero dose position may be determined by the abutment of one of axially extending edges or stops of the number sleeve 80 with at least one or several corresponding stops of the body 10.

The expelling mechanism or drive mechanism 8 as described herein is only exemplary for one of a plurality of differently configured drive mechanisms that are generally implementable in a disposable pen-injector. The drive mechanism as described above is explained in more detail e.g. in WO2004/078239A1, WO 2004/078240A1 or WO 2004/078241A1 the entirety of which being incorporated herein by reference.

Generally, the filling level indicating mechanism as disclosed herein is implementable with any injection device comprising a first member 30 and a second member 10, wherein the first member 30 rotates relative to the second member 10 during and/or for only one of setting of the dose and expelling of the dose and wherein the first member 30 is rotationally locked to the second member 10 during and/or for only one of the other one of setting of the dose and expelling of the dose. _{[IG1]}Any reference to a dose setting member 30 may equally apply to the first member. Moreover, any reference to a body 10 or housing 90 may equally apply to the second member according to the terminology of the appended claims.

The dose setting mechanism 9 as illustrated in FIG. 2 comprises at least the dose dial 12 and the number sleeve 80. As the dose dial 12 is rotated during and for setting of a dose the number sleeve 80 starts to rotate relative to the housing along a helical path as defined by the threaded engagement of its outer thread or helical groove 81 with a correspondingly shaped threaded section at the inside surface of the housing.

During dose setting and when the drive mechanism 8 or the dose setting mechanism 9 is in the dose setting mode the dose setting member 30 rotates in unison with the dose dial 12 and with the number sleeve 80. The dose setting member 30 is threadedly engaged with the piston rod 20, which during dose setting is stationary with regard to the body 10. Accordingly, the dose setting member 30 is subject to a screwing or helical motion during dose setting. The dose setting member 30 starts to travel in proximal direction as the dose dial is rotated in a first sense or rotation or in a dose incrementing direction 4, e.g. in a clockwise direction. For adjusting of or correcting a size of a dose the dose dial 12 is rotatable in an opposite second sense of rotation, hence in a dose decrementing direction 5, e.g. counterclockwise.

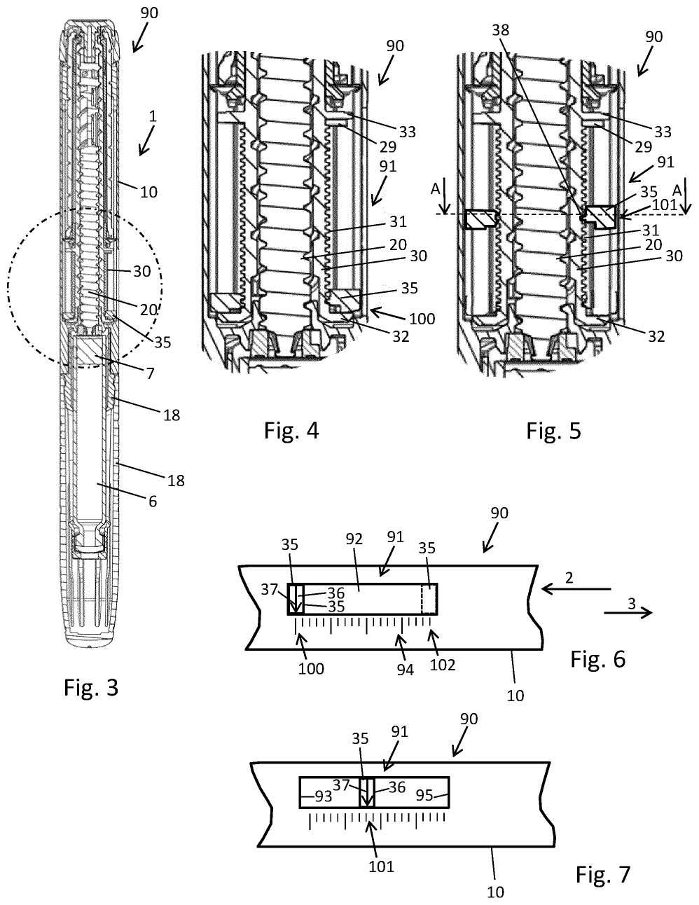

In the example of FIGS. 1-5 a proximal end of the cartridge holder 14 is connectable or is permanently connected to a distal end of the body 10. For this, the cartridge holder 14 may comprise an insert section configured to be inserted in a receptacle provided at the distal end of the body 10. The cartridge holder 14 and the body 10 can be detachably or releasably engaged.

With some examples the cartridge holder 14 and the body 10 are permanently and/or non-releasably or non-detachably engaged, e.g. by a snap-fit interconnection and/or by a force fitting engagement, e.g. provided by an adhesive or by mutual welding of overlapping portions of the cartridge holder 14 and the body 10. With examples, wherein the cartridge holder 14 and the body 10 are non-detachably interconnected the injection device one is implemented as a disposable injection device intended to be discarded in its entirety once the content of the cartridge has been used up or should no longer be used.

The first member 30 and hence the dose setting member 30 may be implemented as a drive sleeve and is configured to rotate in a dose incrementing direction during setting of a dose. For correcting of a dose of the dose setting member may be rotatable in an opposite, hence in a dose decrementing direction relative to the first member and hence relative to the housing 90 or body 10. During dose expelling or dose dispensing the dose setting member 30 can be rotationally locked to the housing.

The filling level indicator 35 of the injection device 1 is engaged with the dose setting member 30. The filling level indicator 35 is further engaged with the elongated housing 90, in particular with the proximally located tubular shaped body 10. In the illustration of FIG. 4 the filling level indicator 35 is located in a start position 100. There, it is in abutment with a first stop 32 provided on the dose setting member 30. The position, in particular the start position 100 of the filling level indicator 35 is discernible from outside the housing through an elongated aperture 91 provided in the sidewall of the housing 90, in particular in a sidewall of the body 10 as indicated in FIGS. 4-7.

In the present example the filling level indicator 35; 135 provides a twofold function. On the one hand it provides the function of a last dose member or last dose nut. On the other hand the filling level indicator 35; 135 is indicative of the filling level of the medicament provided in the cartridge 6. Here, the longitudinal position of the filling level indicator 35; 135 relative to the housing 90 or body 10 directly correlates with the longitudinal position of the piston rod 20 relative to the housing 90 or body 10. The filling level indicator is visually inspectable and/or discernable from outside the housing 90 in each configuration of the injection device.

In FIGS. 4-10 various examples of a filling level indicator 35; 135 are illustrated in connection with one particular example of an injection device 1. Generally, one of the presently disclosed concepts of a filling level indicator 35; 135 is implementable with a variety of different injection devices. With some examples and for the implementation of the filling level indicator 35; 135 there is only required a first member 30, e.g. a dose setting member 30 being rotatable in a dose incrementing direction relative to a second member 10, e.g. a body 10 during setting of a dose and being non-rotatable during dose expelling or dose injection. With other examples the first member is rotationally locked to the second member 10 during and/or for setting of the dose. The first member is then rotatable relative to the second member 20 during and/or for expelling of the dose.

In the presently illustrated non-limiting example the filling level indicator 35 is threadedly engaged with the dose setting member 30. For this, the filling level indicator 35 comprises an inner threaded section 38 threadedly engaged with a correspondingly shaped outer threaded section 31 of the dose setting member 30. The dose setting member 30 comprises a tubular or sleeve-shaped geometrical structure.

The threaded section 31 extends in longitudinal direction on the outside surface of the dose setting member 30. The threaded section 31 is delimited in proximal direction 3 by the second stop 33. The threaded section 31 is delimited in distal direction 2 by the first stop 32. In the presently illustrated example the first stop 32 comprises a radially outwardly extending flange portion integrally formed with the dose setting member 30. Likewise also the second stop 33 is implemented as a radially outwardly extending flange portion. In particular and as illustrated in FIG. 4, there may be provided a stop feature 29 of a distally pointing side of the second stop 33.

The stop feature 34 may comprise a stop surface, wherein the stop surface extends in longitudinal and radial direction. Hence, the surface normal of the stop surface of the stop feature 29 extends in tangential or circumferential direction.

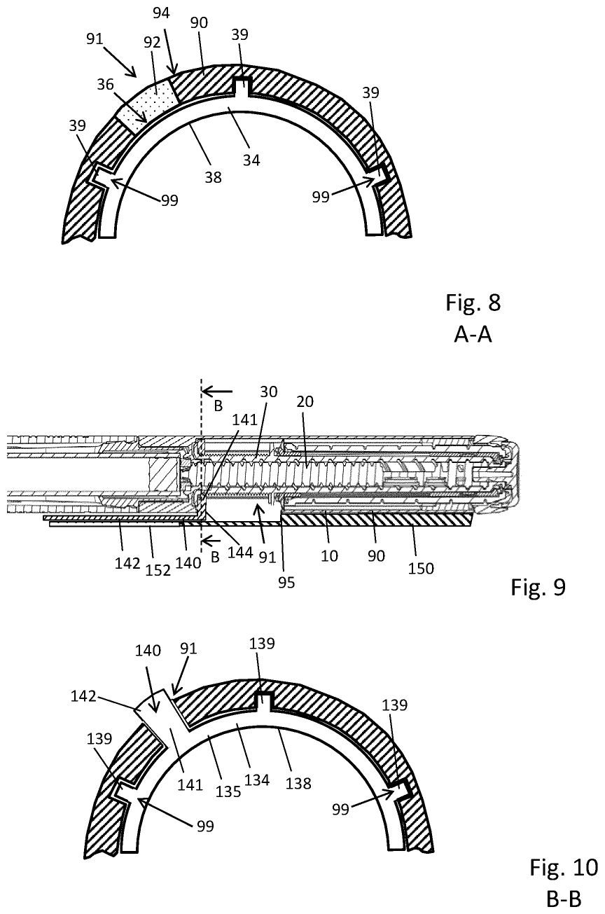

The filling level indicator 35 may comprise a semicircular-shaped half-nut as illustrated in FIG. 8.

The filling level indicator 35 may be either axially engageable with the second stop 33, in particular with the distally directed surface of the flange. Alternatively or additionally the filling level indicator 35 may comprise a stop feature 44, 45 at a circumferential or tangential end configured to abut in tangential or circumferential direction with the stop feature 29 of the second stop 33. In this way, a rather abrupt and well-defined stop configuration can be provided as the filling level indicator 35 reaches an end position 102 as indicated in FIG. 6 by the dashed rectangle.

At least an indicating portion 36 of the filling level indicator 35, in particular an outside facing sidewall portion of the filling level indicator 35 is visually discernible or is visible from outside the housing 90. For this, the housing 90, in particular the body 10, e.g. also representing the second member, comprises an elongated aperture 91 as illustrated in FIGS. 4-8. The elongated aperture 91 comprises a longitudinal elongation that is identical or that substantially matches with the longitudinal elongation between the first stop 32 and the second stop 33 of the dose setting member 30. In this way the longitudinal position of the filling level indicator 35 from the start position 100 towards the end position 102 is always and/or entirely visible. The elongated aperture 91 may be covered or closed by a transparent window 92 as indicated in FIG. 8. In this way, the interior of the body 10 or housing 90 is substantially protected against ingress of humidity and/or particles.

Outside and circumferentially or tangentially offset from the elongated aperture 91 the housing 90 or body 10 comprises at least one or several radial recesses 99 configured to engage with correspondingly shaped radial protrusions 39 of the filling level indicator 35. Here, the radial protrusions 39 extend radially outwardly from an outside surface of the filling level indicator 35. The correspondingly shaped recesses 99 are provided on an inside facing portion of the sidewall of the body 10 or housing 90. An inside of the filling level indicator 35 is provided with a threaded section 38 that is in threaded engagement with an outer counter threaded section 31 of the dose setting member 30.

The recesses 99 provide a sliding track for the filling level indicator 35. The splined engagement of the protrusions 39 with the recesses 99 rotationally locks the filling level indicator 35 to the body 10 or housing 90 thereby enabling a longitudinal sliding movement of the filling level indicator 35 relative to the body 10 in longitudinal direction. A longitudinal sliding displacement of the filling level indicator 35 relative to the body 10 is induced by a rotation of the dose setting member 30 relative to the body 10. Here, the degree of rotation of the dose setting member 30 directly matches with the size of a dose.

On an outside surface of the filling level indicator 35 there is provided an indicating portion 36. The indicating portion 36 is that portion of the filling level indicator 35 that is visible from outside the housing 90.

The filling level indicator 35 further comprises a base portion 34. The base portion 34 is that part of the filling level indicator 35 that is mechanically engaged with the dose setting member 30 and with the housing 90 or body 10.

On the outside surface of the housing 90 or body 10 and along the longitudinal elongation of the longitudinal aperture 91 there is provided a scale 94 provided with numerous marks, text, symbols or numbers that help to indicate the residual amount of medicament provided in the cartridge, when the filling level indicator 35 is located adjacent to a respective portion of the scale 94.

In the configuration as illustrated in FIGS. 4 and 6 the injection device 1 and the drive mechanism 8 are in an initial configuration. The piston rod 20 is in a proximal start position and the amount of medicament inside the cartridge substantially equals the initial filling amount. In this initial configuration there has not been expelled a dose of medicament from the cartridge 6 yet. Accordingly, the filling level indicator 35 is located at or near a first longitudinal end 93 of the elongated aperture 91 thus indicating that the initial filling volume of the cartridge is still available.

As a dose of the medicament is set by rotating the dose setting member 30 relative to the housing 90 the filling level indicator 35 becomes subject to a sliding motion along the elongated aperture 91 towards was the end position 102 as indicated in FIG. 6. The amount of longitudinal displacement is indicated by a pointer 37 of the indicating portion 36 that point towards a particular marker and/or indication of the scale 94 as indicated in FIG. 7.

As the dose has been set and as the filling level indicator 35 has been moved towards the end position 102 the piston rod 20 is still positionally locked to the housing 90 or body 10. It is only during a subsequent dose dispensing or dose expelling procedure that the piston rod 20 is advanced in distal direction relative to the barrel 25 of the cartridge 6 thereby expelling a respective amount of medicament from the cartridge, typically through the injection needle 15 penetrating a piece of an injection site of a patient.

In the intermediate position 101 of the filling level indicator 35 as indicated in FIGS. 5 and 7 the filling level indicator 35 is separated in longitudinal direction from both, the first stop 32 and the second stop 33. The distance to the second stop 33 and hence the distance between the indicating portion 36 and the second longitudinal end 95 of the longitudinal aperture 91 is indicative of the residual amount of medicament that is still available in the cartridge 6. The patient or user of the device is immediately aware of the residual content of the cartridge without being obliged to detach or to remove the protective cap 18 from the cartridge holder 14 to visually inspect the medicament in the cartridge.

In this way and since the filling level indicator 35 is immediately indicative of the remaining filling level of the cartridge the cartridge holder 14 can be made of an opaque material and the medicament located inside the cartridge 6 can be enclosed by a non-transparent and hence opaque cartridge holder. The medicament located inside the cartridge 6 can be protected against electromagnetic radiation when assembled inside the injection device 1 and when received in the cartridge holder 14.

In the further example as illustrated in FIGS. 9 and 10 the filling level indicator 135 also comprises an inner threaded section 138 that is in threaded engagement with the threaded section 31 of the dose setting member 30. Also here, radial protrusions 139 of the filling level indicator 135 are in a sliding engagement with the housing 90 or body 10 of the injection device 1. Also here, the filling level indicator 135 comprises a base portion 134 that is mechanically engaged with both, the housing 90 and the dose setting member 30. Different to the example as described above in connection with FIGS. 4, 5 and 8 the filling level indicator 135 comprises an indicating portion 142 located outside the housing 90 or located outside the body 10.

Here, the filling level indicator 135 comprises a L-shaped rod 140 comprising the indicating portion 142 and a bridging portion 141. The bridging portion 141 is connected to or integrally formed with a longitudinal end of the elongated indicating portion 142. It is also connected to or integrally formed with the base portion 134 of the filling level indicator 135. The bridging portion 141 extends through the elongated aperture 91 in the housing 90 or body 10. In this way the bridging portion 141 is in a longitudinally splined or sliding engagement with the housing 90 or body 10. It is hence rotationally locked to the housing 90 or body 10 through the bridging portion 141. The tangential width of the bridging portion 141 may substantially match or correspond to the tangential size of the elongated aperture 91. In this way, a substantially slack free sliding engagement between the housing 90 or body 10 with the filling level indicator 135 can be provided.

With this example the further protrusions 139 of the filling level indicator 135 may become superfluous. Insofar, the filling level indicator 135 may be implemented also without the radially outwardly extending protrusions 139. Accordingly, also the housing 90 or body 10 may be void of the elongated recessed 99. A longitudinal sliding engagement between the housing 90 and the filling level indicator 135 may be exclusively provided and obtained by the bridging portion 141 extending through the elongated aperture 91 of the housing.

The bridging portion 141 further comprises at least one stop face 144 facing towards the end position 102 and hence facing towards the second longitudinal end 95 of the elongated aperture 91. When approaching the end position 102 the stop surface 144 may abut with the second longitudinal end 95 of the elongated aperture 91 thereby impeding any further displacement of the filling level indicator 135 beyond the end position 102.

Due to the threaded engagement between the filling level indicator 135 and the dose setting member 30 a further dose incrementing rotation of the dose setting member for setting of a dose that would exceed the amount of medicament left in the cartridge 6 can be effectively prevented.

Insofar, the filling level indicator 135 fulfills a twofold function. On the one hand the filling level indicator 135 visually illustrates the residual amount of medicament provided in the cartridge 6. On the other hand the filling level indicator 135 provides an end stop preventing dialing or setting of a dose exceeding the residual amount of medicament provided in the cartridge.

With the example of FIGS. 9 and 10 the elongated aperture 91 of the housing 90 is void of a covering window 92. Rather, a respective portion of the housing 90 may be encapsulated or provided with an outer housing 150 as illustrated in FIG. 9. The outer housing 150 at least covers the aperture 91. The outer housing 150 also covers at least a portion or the entirety of the rod-shaped and elongated indicating portion 142 of the filling level indicator 135. The outer housing 150 may cover the entirety of the L-shaped rod 140 in longitudinal direction and in any available position of the filling level indicator 135 relative to the housing 90 or body 10.

The outer housing 150 may further comprise a transparent portion 152. The transparent portion 152 may comprise a transparent window in the outer housing 150. The entirety of the elongated indicating portion 142 or only a portion thereof may be discernible in the transparent portion 152 of the outer housing 150. One of the indicating portion 142 and the transparent portion 152 is or may be provided with a scale whereas the other one of the indicating portion 142 and the transparent portion 152 is provided with a pointer pointing towards the scale.

The outer housing 150 may be opaque at least in a region in which it covers the elongated aperture 91 of the housing 90 or body 10. In this way, the internal structure or the interior of the dose setting mechanism 9 and/or of drive mechanism 8 can remain covered and invisible from the outside of the housing 90 or outer housing 150.

### LIST OF REFERENCE NUMBERS

1 injection device
2 distal direction
3 proximal direction
4 dose incrementing direction
5 dose decrementing direction
6 cartridge
7 bung
8 drive mechanism
9 dose setting mechanism
10 second member
11 trigger
12 dose dial
13 dosage window
14 cartridge holder
15 injection needle
16 inner needle cap
17 outer needle cap
18 protective cap
19 protrusion
20 piston rod
21 bearing
22 first thread
23 pressure foot
24 second thread
25 barrel
26 seal
28 threaded socket
29 stop feature
30 first member
31 threaded section
32 stop
33 stop
34 base portion
35 filling level indicator
36 indicating portion
37 pointer
38 threaded section
39 protrusion
40 spring
50 dose tracker
51 tracking stop feature
60 clutch
62 insert piece
64 stem
80 number sleeve
81 groove
90 housing
91 aperture
92 window
93 longitudinal end
94 scale
95 longitudinal end
99 recess
100 start position
101 intermediate position
102 end position
134 base portion
135 filling level indicator
138 threaded section
139 protrusion
140 rod
141 bridging portion
142 indicating portion
144 stop face
150 outer housing
152 transparent portion

1.-15. (canceled)

16. An injection device for setting and injecting a dose of a medicament, the injection device comprising:
an elongated housing extending along a longitudinal direction, the elongated housing being configured to accommodate a cartridge;
a first member movably arranged inside the elongated housing for setting of the dose;
a second member;
a piston rod configured to advance in a longitudinal distal direction and operable to exert a distally directed pressure onto a bung of a cartridge for expelling the dose, wherein during one of setting the dose and expelling the dose the first member is rotatable relative to the second member, and wherein during the other one of setting the dose and expelling the dose the first member is rotationally locked to the second member; and
a filling level indicator threadedly engaged with one of the first member and the second member and wherein the filling level indicator is rotationally locked and longitudinally slidingly engaged with the other one of the first member and the second member, wherein the filling level indicator is movable in a longitudinal direction at least from a start position to an end position relative to at least one of the first member and the second member, and wherein the filling level indicator is visible from outside the elongated housing when the filling level indicator is in the start position.

17. The injection device of claim 16, wherein the filling level indicator is visible from outside the elongated housing when the filling level indicator is in the end position.

18. The injection device of claim 16, wherein the filling level indicator is visible from outside the elongated housing in any position between the start position and the end position.

19. The injection device of claim 16, wherein one of the first member and the second member comprises a first stop and a second stop, wherein the first stop and the second stop are separated from each other along the longitudinal direction.

20. The injection device of claim 19, wherein the filling level indicator engages the second stop when in the end position, and wherein the filling level indicator is operable to prevent a further movement of the first member relative to the second member at least with regard to one direction of movement when the filling level indicator is engaged with the second stop.

21. The injection device of claim 16, wherein the elongated housing comprises an elongated aperture extending along the longitudinal direction, and wherein at least an indicating portion of the filling level indicator is visible through the elongated aperture.

22. The injection device of claim 21, wherein the elongated aperture is covered or closed by a transparent window.

23. The injection device of claim 16, wherein the elongated housing comprises an elongated aperture extending along the longitudinal direction, and wherein at least an indicating portion of the filling level indicator is located outside the elongated housing.

24. The injection device of claim 23, wherein the filling level indicator comprises a L-shaped rod comprising the indicating portion and a bridging portion, wherein the bridging portion extends through the aperture.

25. The injection device of claim 23, further comprising an outer housing covering or enclosing at least a portion of the elongated housing in a region of the filling level indicator, wherein the outer housing comprises a transparent window overlapping at least with the indicating portion of the filling level indicator.

26. The injection device of claim 16, wherein the elongated housing comprises a cartridge holder configured to accommodate the cartridge, wherein the cartridge holder is opaque.

27. The injection device of claim 21, wherein the elongated housing comprises a scale extending along the elongated aperture, and wherein the indicating portion comprises a pointer pointing towards the scale.

28. The injection device of claim 16, wherein the filling level indicator comprises a threaded section and at least one of a radial protrusion and a radial recess.

29. The injection device of claim 28, wherein the threaded section is engaged with a correspondingly shaped counter threaded section of the one of the first member and the second member.

30. The injection device of claim 28, wherein the at least one of the radial protrusion and the radial recess is in a longitudinal sliding engagement with a correspondingly shaped radial recess or the radial protrusion of other one of the first member and the second member.

31. The injection device of claim 16, wherein the first member is movable relative to the second member only during one of setting the dose and dispensing the dose.

32. The injection device of claim 16, further comprising the cartridge arranged inside the elongated housing, the cartridge being filled with the medicament.

33. A method for setting and injecting a dose of a medicament using an injection device, the method comprising:
setting the dose by rotating a first member relative to the second member, wherein the first member is movably arranged inside the elongated housing for setting the dose;
advancing a piston rod in a longitudinal distal direction to exert a distally directed pressure onto a bung of a cartridge for expelling of the dose by rotationally locking the first member to the second member; and
threadedly engaging a filling level indicator with one of the first member and the second member, wherein the filling level indicator is rotationally locked and longitudinally slidingly engaged with the other one of the first member and the second member, the filling level indicator being movable in a longitudinal direction at least from a start position to an end position relative to at least one of the first member and the second member, and wherein the filling level indicator is visible from outside the elongated housing when the filling level indicator is in the start position.

34. The method of claim 33, further comprising:
engaging by the filling level indicator the second stop when in the end position; and
preventing by the filling level indicator a further movement of the first member relative to the second member at least with regard to one direction of movement when the filling level indicator is engaged with the second stop.

35. The method of claim 33, further comprising:
bringing the filling level indicator in the end position in which the filling level indicator is visible from outside the elongated housing.
